(19) United States
(12) Patent Application Publication
(10) Pub. No.: US 2011/0009748 A1
(43) Pub. Date: Jan. 13, 2011

(54) TRANSPERINEAL PROSTATE BIOPSY SYSTEM AND METHODS

(75) Inventors: Damien GREENE, Newcastle upon Tyne (GB); Ofri VAISMAN, Doar-Na Emek Soreq (IL); Natan KARMON, Doar-Na Misgav (IL)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: GALIL MEDICAL LTD., Yokneam (IL)

(21) Appl. No.: 12/814,007

(22) Filed: Jun. 11, 2010

Related U.S. Application Data
(60) Provisional application No. 61/213,460, filed on Jun. 11, 2009.

(51) Int. Cl. A61B 8/13; A61B 18/02; A61B 10/02

(52) U.S. Cl. 600/439; 600/443

(57) ABSTRACT

Method and systems for performing ultrasound guided transperineal prostate biopsies. A template having a plurality of apertures and attached to a transrectal ultrasound probe is used for planning and guiding the biopsy. The template is placed against the patient's perineum and transverse ultrasound images having a projected template image are displayed. Planned biopsy locations are marked on the projected aperture image and biopsy samples are obtained through the corresponding template apertures. Sagittal ultrasounds may be viewed to ensure the correct depth of the biopsy needle during the biopsy. Information about the location of the biopsy sample can be recorded by identifying the template aperture through which the biopsy was obtained. Stored biopsy location information can be used for planning later treatments which can use a template having the same set of apertures.

### PRIORITY CLAIM

The present application claims priority to U.S. Provisional Patent Application No. 61/213,460, entitled TRANS RECTAL PROSTATE BIOPSY PLANNING AND MANAGEMENT ALGORITHM, and filed Jun. 11, 2009, the disclosure of which is herein incorporated by reference in its entirety.

### BACKGROUND

Screening processes for prostate cancer include monitoring blood levels of prostate specific antigen (PSA) and digital rectal examination. When these tests indicate a possible abnormality, further tests can include imaging such as transrectal ultrasounds. However, while these examinations can indicate the possibility of prostate cancer, a definitive diagnosis requires tissue biopsy.

Due to the location of the prostate immediately anterior to the rectum, one method of biopsying the prostate includes passing biopsy needles through the rectal wall and into the prostate at various locations. This technique is effective at obtaining biopsy samples which can then be analyzed to determine whether they include cancerous cells. During this procedure, the physician notes the location within the prostate from which the samples are taken.

When prostate cancer is identified, there are various therapies available for treatment of the cancer, and often it is possible to completely cure the patient. However, because of the location of the prostate, the therapies can be associated with varying rates of unwanted side effects, including incontinence and impotence. Furthermore, the choice of which type of therapy is preferred for an individual patient depends, among other things, upon the size and location of the tumor.

Cancer treatment therapies include surgery (such as transurethral resection or open prostatectomy), radiation (external beam or brachytherapy), and cryotherapy. Typical cryotherapy methods include localized cancer treatment using needles which can be inserted into the prostate through the perineum. The tissue surrounding the tip of the cryotherapy needle is reduced to very low temperatures, forming an ice ball and destroying the tissue in a small area. This method of treatment therefore requires accurate placement of the cryotherapy probes at the location of the tumor. In order to achieve this degree of accuracy in placement of the cryoprobes, the physician placing the cryotherapy probes must know the exact location of the tumor.

### SUMMARY

Embodiments of the invention include methods and systems for planning and performing transperineal prostate biopsies. The biopsies may be performed using a system including a transrectal ultrasound probe attached to a template grid having a plurality of apertures, a visual display, a biopsy control unit in electrical communication with the ultrasound probe and the visual display, and one or more biopsy needles. The biopsy control unit may be a central processing unit capable of processing ultrasound image data, registering the template's location relative to the ultrasound probe, and sending data to the display to create a transverse ultrasound image of the prostate including a projected template image having projected apertures. The system may also include a user interface, allowing a user to mark one or more projected apertures to be used for performing the biopsy. The biopsy needles are sized for insertion through the apertures and into the prostate when the template is placed against the patient's perineum. The template apertures may be spaced to allow biopsy sampling of the entire prostate.

The system may also include a digital storage medium, or may be designed for connection to a digital storage medium, so that information about the locations at which the biopsies were performed may be stored and later retrieved. This stored biopsy information may then be used to assist in the performance of localized prostate treatment, such as cryotherapy. For example, a biopsy sample may be found to be positive for cancer. The stored information about this sample, such as the aperture location through which it was obtained and the depth of the sample within the prostate, may be retrieved later, and a cryotherapy needle may be accurately placed in the same location by placement through the same template aperture and at the same depth as determined using the stored information.

Methods of the invention include placing the template against the patient's perineum, acquiring a transverse ultrasound image of the prostate in a first plane using the transrectal ultrasound probe, registering the template to display projected template apertures on the transverse ultrasound image, marking a projected template aperture as a planned biopsy location, and obtaining a sagittal ultrasound image of the prostate. The sagittal ultrasound image may be in the same plane as the biopsy needle, so that the depth of the needle within the prostate may be seen. The method further includes inserting a biopsy needle through the template aperture corresponding to the marked projected template aperture and obtaining a biopsy sample in the first plane. Identification of the template aperture used for obtaining the biopsy sample may be recorded. The ultrasound images may also be recorded including the transverse ultrasound and/or the sagittal ultrasound images. The recorded sagittal ultrasound image may be acquired when the biopsy needle is in position to obtain the biopsy sample, so that the depth of the sample location at the time of biopsy can be identified.

In some embodiments, the projected aperture is marked with a first color to indicate that it is a planned biopsy location. The marking of the projected aperture may then be changed to a second color after the biopsy sample has been obtained at that location. The transverse ultrasound image may also be marked to identify other aspects of the patient's anatomy, such as the location of the prostate gland, the prostatic urethra, and/or the colon.

Methods of the invention may also include performing cryotherapy at the location of the biopsy sample including inserting a cryotherapy needle through the template aperture corresponding to the marked and stored projected template aperture.

In some embodiments, registration of the template includes inserting two or more biopsy needles through two or more separate template apertures, displaying the transverse ultrasound image, marking the locations of each biopsy needle on the displayed transverse ultrasound image, identifying the template apertures through which each biopsy needle was inserted, and calculating the projected template aperture locations.

In some embodiments, methods of the invention include obtaining a second biopsy sample in a second transverse plane. In addition to the steps described above, the method includes acquiring a second transverse ultrasound image of the prostate in a second plane using the ultrasound probe, marking a second projected template aperture as a second planned biopsy location on the second transverse ultrasound image, inserting a second biopsy needle through the template aperture corresponding to the marked projected template aperture to the biopsy location in the second plane, obtaining a second biopsy sample in the second plane using the second biopsy needle, and recording an identification of the second aperture for the second biopsy sample.

Alternatively, in some embodiments, methods of the invention include obtaining a second biopsy sample in the same transverse plane as the first biopsy sample. After obtaining a first biopsy sample as described above, the method includes marking a second projected template aperture as a planned biopsy location in the first plane, inserting a second biopsy needle through the template aperture corresponding to the second marked template aperture, obtaining a second biopsy sample in the first plane using the second biopsy needle, and recording an identification of the template aperture used for obtaining the second biopsy sample.

In some embodiments, the biopsy is performed using a biopsy gun. In such embodiments, the method may include advancing the biopsy needle, stopping insertion of the biopsy needle at a location superficial to the first plane (that is, less deep or closer to the surface of the patient's body), and firing the biopsy gun to project the biopsy needle into the first plane.

In some embodiments, methods of the invention include placing the template against the patient's perineum, acquiring a transverse ultrasound image of the prostate in a first plane, registering the template to display projected template apertures on the transverse ultrasound image, marking two or more projected template apertures as a planned biopsy locations, inserting biopsy needles through each of the template apertures corresponding to the marked projected template apertures, acquiring sagittal ultrasound images of the prostate in the plane of each biopsy needle, obtaining biopsy samples using the biopsy needles, recording an identification of the template apertures used for obtaining the biopsy samples, acquiring a transverse ultrasound image of the prostate in a second plane using the ultrasound probe, and repeating each of the steps in the second plane.

### BRIEF DESCRIPTION OF THE DRAWINGS

FIG. 1 is a perspective view of an ultrasound and template system according to embodiments of the invention;

FIG. 2 illustrates an ultrasound and template system and the formation of images of a patient's prostate at various depths according to embodiments of the invention;

FIG. 3 illustrates and ultrasound and template system in use during a prostate biopsy according to embodiments of the invention;

FIG. 4 is a system diagram according to embodiments of the invention

FIG. 5 is a transverse ultrasound image of a prostate including a projected template;

FIG. 6 is a sagittal ultrasound image of a prostate.

### DETAILED DESCRIPTION

Embodiments of the invention include an imaging device used in combination with a template for planning and executing transperineal biopsies of the prostate. The imaging device may be a biplanar transrectal ultrasound (TRUS) which provides transverse and sagittal images of the prostate on a visual display. The template has a set of apertures and may be connected to the imaging device. The template may placed in a location close to or abutting the patient's perineum, such that biopsy needles may be passed through the apertures and into the prostate at a desired location as determined by the physician using the ultrasound images.

Prior to taking the biopsy samples, the biopsy procedure may be carefully planned by the physician. The locations from which samples will be obtained may be planned using the visual display, the template, and an associated biopsy control unit. The biopsy control unit may register the template and display a projected template onto transverse prostate images on the visual display, showing the locations at which biopsy needles passing through each of the apertures of the template would intersect the plane of the image. The physician may then use the images including the projected template to plan the biopsy procedure. In some embodiments, the system may further include a user interface which allows the physician to select and mark the projected template apertures to be used for biopsy. The system may further allow the physician to change the markings at each aperture once the biopsy is completed, in order to facilitate the biopsy process.

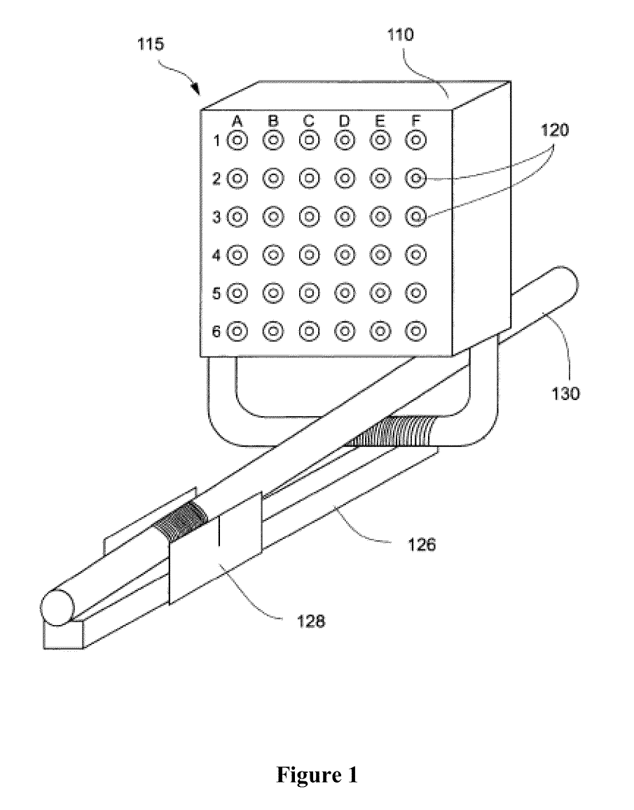

FIG. 1 is a simplified schematic of an apparatus comprising an ultrasound probe 130 and a template 115 for guiding insertion of a plurality of biopsy needles into a patient's body. As shown in FIG. 1, an ultrasound probe 130 is provided for insertion into the patient's rectum, ultrasound probe 130 being received within a housing element 128. A template 115 is connected to housing element 128 by means of a connecting arm 126. As shown, template 115 is in the form of a plate 110 having a net of apertures 120, each aperture serving for insertion of a biopsy needle therethrough. In some embodiments, the distance between each pair of adjacent apertures 120 is between about 2 millimeters and about 5 millimeters. The template may be the same, or may have the same relative aperture locations, as a template used for cryoablation in the same patient at a later time. That is, the cryotherapy template apertures may be spaced the same and may be located in the same position relative to the ultrasound probe as the biopsy template apertures. In this way, a cryotherapy template may be used by the physician to easily deliver the cryoprobe to the same location as that from which a positive biopsy sample was obtained by delivering the cryotherapy probe through the same or corresponding aperture in the cryotherapy template.

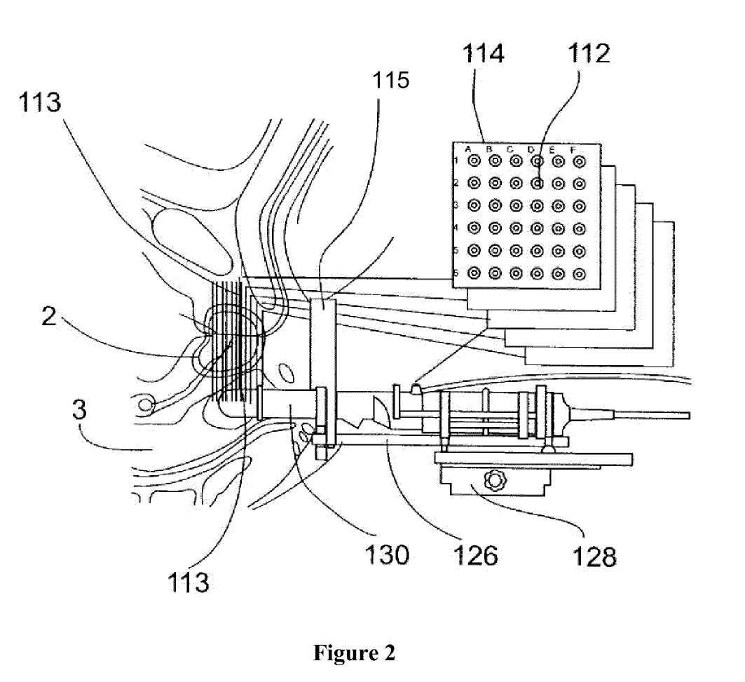

FIG. 2 shows an ultrasound probe 130 introduced to a specific depth 113 within the patient's rectum 3. The ultrasound probe 130 and template 115 are electrically connected to a biopsy control unit. The biopsy control unit registers the ultrasound image to provide a projected template 112 as a net of marks on the obtained ultrasound image 114, the net of marks on cross sectional image 114 being accurately correlated to the net of apertures 120 on template 115. The set of images 114 provides a three dimensional grid of the prostate. Such three-dimensional grid is then used for planning the biopsy procedure.

Thus, marks or apertures 120 on image 114 identify the exact locations of where biopsy needles would pass through the plane of the ultrasound image after insertion through apertures 120 into the patient's prostate 2. Image 114 relates to a specific depth of penetration 113 of the biopsy needles into the prostate 2. Thus, each of images 114 relates to a specific plane perpendicular to the axis of penetration of the biopsy needles. The system can further display sagittal ultrasound images so that during the biopsy procedure, advancement of the needle can be seen, such that samples are taken at the depth of penetration shown in the transverse cross section 114.

Biopsies may be planned and obtained at multiple depths within the prostate 2. The biopsy may be performed by taking samples first at a more superficial depth, such as at the apex, then at a deeper depth, such as mid-gland, and lastly at the deepest depth, such as at the base of the prostate 2. For example, the introduction of a biopsy needle along a given axis of penetration to a first depth may effectively sample tissue at a first depth such as at the apex of the prostate 2, while introduction of the biopsy needle to a second depth may sample tissue at a second depth such as at the base of the prostate 2.

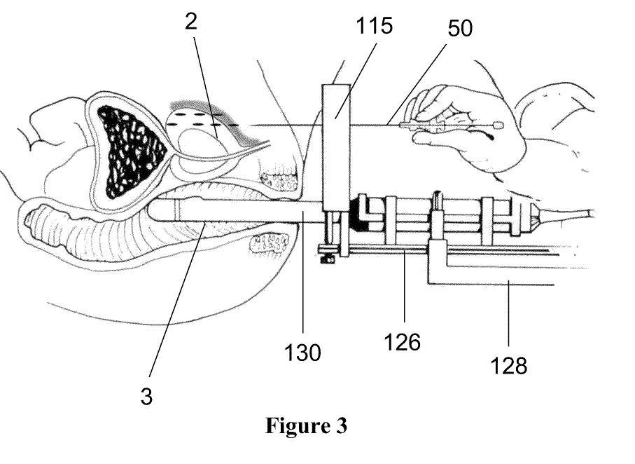

FIG. 3 shows the insertion of a biopsy needle 50 through an aperture 120 of a template 115 into the prostate 2 of a patient. A plurality of biopsy needles 50 may be sequentially inserted through the apertures 120 of the template 115 into the patient's prostate 2, wherein each needle 50 is introduced to a specific depth. The doctor performing the procedure may observe the depth of penetration of the biopsy needle 50 by simultaneously viewing an ultrasound image of the prostate in the sagittal plane. In some embodiments, the depth of penetration may be set by advancing the biopsy needle 50 until it abuts the template 115.

The systems and methods presented in FIGS. 1-3 enable diagnostic mapping of areas to be biopsied within a prostate 2, and enable guiding a plurality of biopsy needles 50 into a prostate 2 in such a manner that the needles 50 are placed according to the planned biopsy areas so mapped. Furthermore, the locations of the biopsy samples can be recorded according to the template aperture 120 and sample depth as determined by the physician using the sagittal ultrasound view, and this information can be retained, along with the ultrasound images, for later use for planning and performing localized cancer treatment if necessary.

Any standard biopsy needles 50 useful for transperineal ultra-sound guided prostate biopsy may be used in embodiments of the invention. In some embodiments, the biopsy needle 50 may be included in a biopsy gun. The biopsy needle 50 should have sufficient rigidity and length to allow it to be inserted through the perineum and into the prostate 2. Furthermore, it should be sized to allow it to pass smoothly through the apertures 120 of the template 115. That is, the biopsy needle 50 should not have a circumference which is so much smaller than the aperture 120 that it moves about within the aperture 120, rather than being held in position within the aperture 120 while still allowing the biopsy needle 50 to be advanced and retracted. In some embodiments, the biopsy needles 50 may include a scale for observing the depth of penetration into the prostate.

The template 115 may be any apparatus comprising a plurality of apertures 120 sized to accommodate and to direct insertion of one or more biopsy needles 50 into a body. Appropriate templates are available from Galil Medical, Ltd., Yokneam, Israel. The template 115 may be a rectangular object constructed of metal or plastic and comprising a regular two-dimensional array of apertures 120 of standard size and parallel orientation, as shown in FIG. 1, for example. The template 115 may include a coordinate system to label the apertures 120 or other labeling system. For example, it may include numerical and/or alphabetical markings to identify the apertures 120. The template 115 may be any object comprising a plurality of apertures 120 through which one or more biopsy needles 50 may be inserted, the apertures 120 serving to direct or limit insertion direction and/or depth of insertion of needles 50 inserted through the apertures 120. In some embodiments, the apertures 120 are 17 gauge holes. Apertures 120 of template 115 may be designed to direct a plurality of biopsy needles 50 inserted therethrough into body tissues along substantially parallel paths. Templates 115 contemplated by the present invention may be of any shape and may comprise non-regular aperture arrays and non-parallel apertures. The template 115 may be formed to fit and securely attach to a frame which is rigidly connected to the housing 128, thereby providing stability and a fixed position of template 115 with respect to other parts of the apparatus and with respect to a patient.

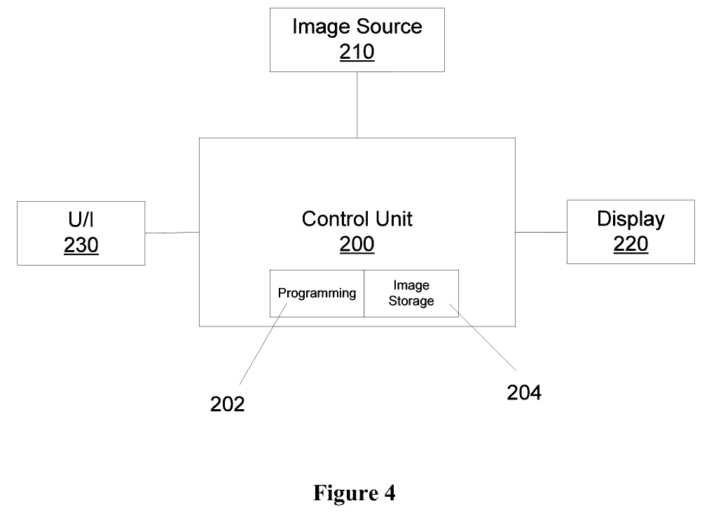

FIG. 4 provides a schematic diagram of a system according to embodiments of the invention. The system includes a biopsy control unit 200 in electrical communication with an image source 210, a visual display 220 and a user interface 230.

The image source 210 may be an ultrasound probe, such as a biplanar transrectal ultrasound probe. However, images may alternatively or additionally be provided by other image sources such as MRI or CT. The image source provides data corresponding to transverse images to the biopsy control unit 200, which are then displayed on the visual display 220 along with the template projection 112. The image source 210 also provides data corresponding to sagittal images to the biopsy control unit 200, and the sagittal image is provided on the display 220 and may be used to determine depth of needle penetration during the biopsy.

Data received by the biopsy control unit 200 may be processed and output to any standard visual display 220, such as a computer monitor or television type screen. In some embodiments, the visual display may be a touch screen and may also function as a user interface 230. For example, the visual display may include an on-screen virtual keyboard. Display 220 may be a flat panel display such as LCD, or may be a CRT or plasma display, a stereoscope display device, or other graphic display. In some embodiments, the display 220 may be mounted on the biopsy control unit 200 such as by an articulated arm. In such embodiments, the biopsy control unit 200 may be mounted on lockable wheels, such that, along with the display 220 and user interface 230, it forms a mobile workstation. The workstation may be positioned next to the procedure table when in use.

User interface 230 may comprise any interface equipment such as a keyboard, mouse, and/or stylus pen operable to receive user input. Optionally, a plurality of user interfaces 230 may be used. User interface 230 may enable a user to characterize portions of displayed images, such as to identify or outline organs or biopsy targets. User interface 230 will also typically enable a user to input command decisions or preferences. It may further allow the user to highlight or mark planned biopsy locations on the projected template 112 on the display 220. The planned biopsy locations may then be marked differently after a biopsy is performed at that location. For example, the planned biopsy site at a projected aperture location on the display 220 may be marked by the user with a first color, such as red. After the biopsy is performed at that location through the specified template aperture 120, the user may mark the projected aperture on the display 220 with a second color, such as green. In this way, the user can easily tell which apertures 120 of the template 115 need to be used for the biopsy, and which apertures 120 have already been used for biopsy.

The biopsy control unit 200 may be any programmable computer processor capable of processing image date and user commands. The biopsy control unit 200 receives and processes image data received from an image source 210 and transmits it to the display 220 where the image 114 may be shown in combination with the projected template 112 image including projected apertures which may be shown as circles or dots, for examples. As such, the biopsy control unit 200 includes programming 202 for creating a visual image 114 on the display 220 which includes the prostate image as well as the projected template image 112. The biopsy control unit 200 may further receive user input to alter the image 114 as described as above, such as marking or drawing on the image 114. It may therefore also include a graphics program to receive the user input and provide the corresponding transmission to the display 220. The biopsy control unit 200 may further include memory 204 for storing images 114 and user input. Alternatively, the biopsy control unit 200 may include a port for connection to an external memory storage device. These stored images 114 and other data input may later be retrieved and used for cryotherapy treatment planning. The biopsy control unit may include ports for connection to the visual display 220, the user interface 230 and/or the image source. In addition, the biopsy control unit may include ports for connecting to a network, such as the internet. In some embodiments, the biopsy control unit may be connected to, or connectable to, a printer. The printer may be used for printing biopsy planning or procedure reports. In some embodiments, the printer may be included with the biopsy control unit 200 as part of a mobile work station.

In practice, the systems described herein may be used for planning and performing a prostate biopsy. A patient in need of a prostate biopsy is put under general or local anesthesia and is placed in the lithotomy position on a procedure table. A system including an ultrasound probe 130 and template 115, such as the system of FIG. 3, is in a secure position, such as affixed to the procedure table. The ultrasound probe 130 and template 115 system are positioned appropriately relative to the patient, with the template 115 against or near the patient's perineum and the positioned to enter the rectum. In this way, with both the patient and the probe 130 and template 115 system are securely positioned, such as by attachment to the table, so that their relative positions are known throughout the procedure. A fixed relationship (or other known positional relationship) between the probe 130 and template 115 simplifies registration of images provided by the probe 130.

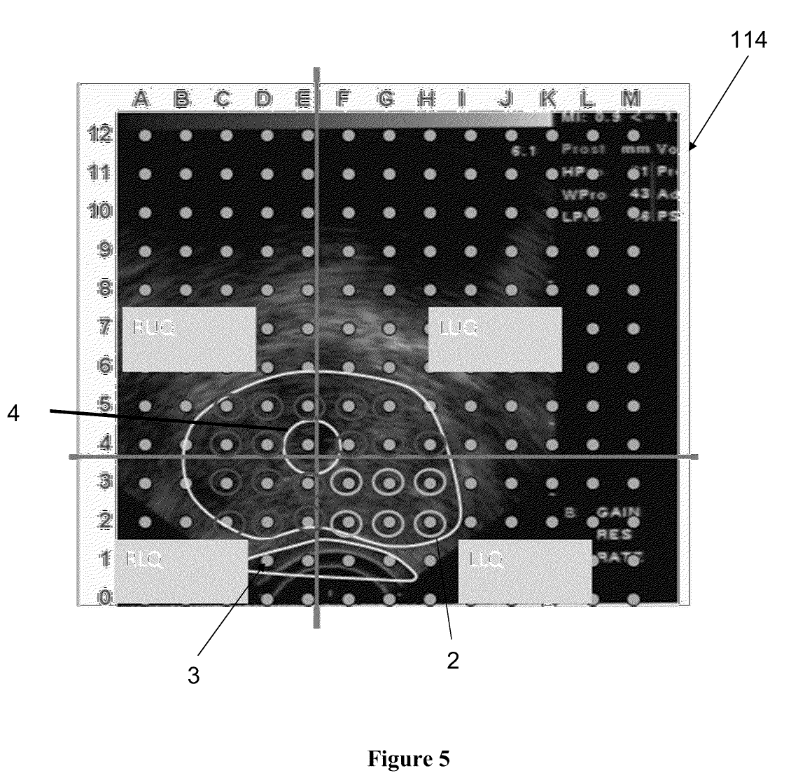

The ultrasound probe 130 may be advanced manually by the physician. Alternately, the ultrasound probe system may include a mechanical advancement mechanism, such as an ultrasound probe stepper system. In such embodiments, the probe may be may be advanced and withdrawn smoothly by the stepper. The stepper system includes a position gauge indicating ultrasound probe 130 position, which facilitates placement of the ultrasound probe 130 during the procedure. When the ultrasound probe 130 is activated, image data is transmitted to the biopsy planning unit 200 which processes the data and transmits it to the visual display 220. The physician may obtain transverse and/or sagittal images during the procedure. Because the position of the template 115 relative to the ultrasound probe is known and fixed, the biopsy planning unit 200 may further process the image data to register the projected template image 112 onto the visual display 220 along with, or superimposed upon, the ultrasound images 114. The template 115 may be registered by inserting one, or preferably two, biopsy needles 50 through the template 115 at a selected prostate plane, such as at the widest portion of the prostate 2. A transverse ultrasound image 114 is then captured, the needle 50 locations are marked on the display 220 by the physician along with an identification of the aperture 120 through which each needle 50 was placed. The biopsy planning unit 200 then processes this information to register the template 115 location relative to the ultrasound probe 130 in order to calculate and produce a projected template 112 on the display 220. An example of a transverse ultrasound image 114 including a projected template 112 is shown in FIG. 5.

In order to plan the biopsy, the physician positions the ultrasound probe 130 at a first position to obtain a first transverse ultrasound image 114. The physician may then delineate or mark the displayed image 114, using the user interface 230, to identify the relevant anatomy. In FIG. 5, the physician has outlined the prostate 2, urethra 4 and rectum 3, and has labeled the right upper quadrant (RUQ), left upper quadrant (LUQ), right lower quadrant (RLQ) and left lower quadrant (LLQ) of the prostate 2.

The physician may then select one or more apertures 120 for placement of a biopsy needle 50 and may mark the projected apertures on the visual display 220 through the user interface 230. In FIG. 5, the physician has marked the projected apertures of the right and left upper quadrants and right lower quadrant for biopsy by encircling them with red, which may be seen as a dark grey in FIG. 5.

The physician may proceed to biopsy the prostate at the selected location by inserting a biopsy needle 50 through the corresponding apertures 120 on the template 115 and advancing the biopsy needle 50 to the plane at which the ultrasound was taken. The physician may take a sagittal image of the prostate 2, in the plane of the advancing needle 50, to observe and confirm that the needle 50 has been advanced to the appropriate location. In some embodiments, the biopsy gun may take samples 24 mm ahead of, or deeper as, the tip of the biopsy gun. In such embodiments, the biopsy gun may be stopped at a position which is short of the transverse plane by an amount equal to the distance by which the biopsy needle is propelled forward when fired, so that the biopsy sample is taken within the selected transverse plane. The process is then repeated, using a new biopsy needle 50, for each of the selected template apertures 120 within the plane.

The physician may select additional planes from which to obtain biopsy samples. For example, the physician may adjust the position of the ultrasound probe 130 to display a transverse ultrasound image in a second plane. Projected apertures may again be marked on the display 220 and the biopsies performed in the second plane in the same manner as in the first plane.

After each biopsy sample is obtained, the physician may change the demarcation of the projected aperture on the visual display 220 to indicate that the sample has been completed at this location. In FIG. 5, the projected apertures of the left lower quadrant are encircled in green, which appears as a light grey in FIG. 5 to indicate that biopsy samples have been removed at these locations.

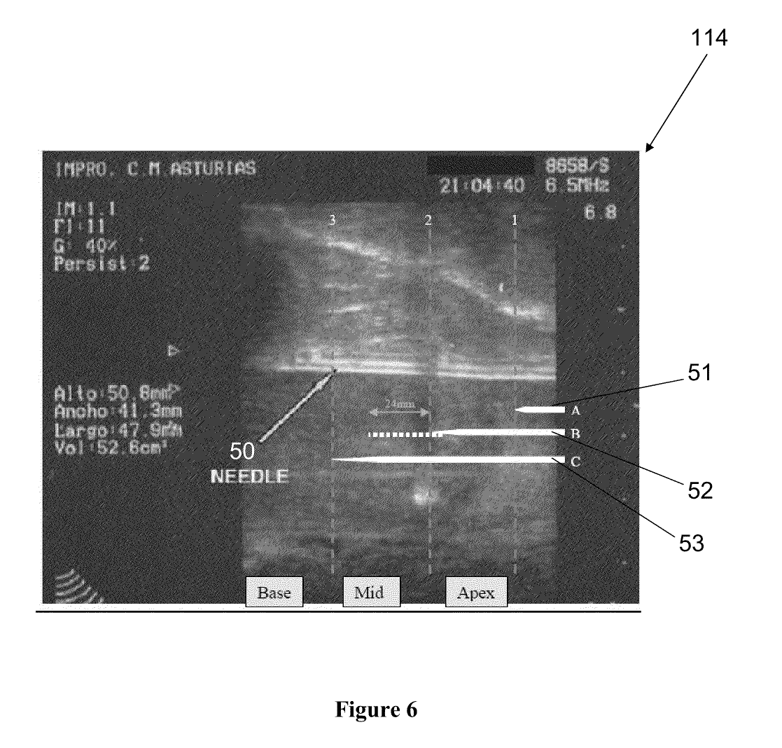

An example of a sagittal image showing advancement of the biopsy needle 50 within the prostate 2 is shown in FIG. 6. As shown, the physician can visualize the tip of the biopsy needle 50 and therefore can see the depth of penetration of the needle. For demonstration purposes, FIG. 6 also shows three representative biopsy needles 51, 52, 53, which were added to the ultrasound image to demonstrate how ultrasound needles 50 may be placed at various depths using a sagittal ultrasound image. Representative needle 51 is shown at the apex, while representative needle 52 is in the mid prostate, and representative needle 53 is in the base. Furthermore, representative needle 52 shows how, when the biopsy gun is fired, the needle may advance forward an additional 24 mm. The representative needle 52 is therefore positioned 24 mm short of the location from which a sample would actually be obtained.

After each biopsy sample is obtained, the needle 50 is withdrawn and the sample is labeled to identify the aperture 120 from which the sample was obtained. The sample may be further labeled to identify the depth of the location from which the sample was obtained, as noted by the physician using a sagittal ultrasound image.

Ultrasound planning images, as well as images taken during the biopsy procedure, may be stored by the biopsy planning unit memory 202 or in an external memory device for later analysis of the tumor location in case any of the samples is positive for cancer. The images may also provide a reference for comparison during future treatment procedures, to ensure that cryotherapy needles or cryoprobes are placed in the same locations as those from where the biopsy samples were taken. The cryotherapy needles may be connected to a cryotherapy control unit to control the delivery of the cryotherapy. An example of a cryotherapy system which may be used is the Presice® cryoablation system, available from Galil Medical, Ltd., Yokneam, Israel. In some embodiments, the same system may be used for both transperineal prostate biopsy and cryotherapy.

In the foregoing detailed description, the invention has been described with reference to specific embodiments. However, it may be appreciated that various modifications and changes can be made without departing from the scope of the invention as set forth in the appended claims. Thus, some of the features of preferred embodiments described herein are not necessarily included in preferred embodiments of the invention which are intended for alternative uses.

1. A method for performing a transperineal biopsy of a patient's prostate, comprising:
a) placing a template having a plurality of apertures against the patient's perineum, wherein the template is attached to a transrectal ultrasound probe;
b) acquiring a transverse ultrasound image of the prostate at a first plane using the ultrasound probe;
c) registering the template to display projected template apertures on the transverse ultrasound image;
d) marking a projected template aperture as a planned biopsy location;
e) acquiring a sagittal ultrasound image of the prostate;
f) inserting a biopsy needle through the template aperture corresponding to the marked projected template aperture;
g) obtaining a biopsy sample in the first plane using the biopsy needle; and
h) recording an identification of the template aperture used for obtaining the biopsy sample.

2. The method of claim 1, further comprising recording the transverse ultrasound.

3. The method of claim 1, further comprising recording the sagittal ultrasound.

4. The method of claim 3, wherein the recorded sagittal ultrasound image is acquired when the biopsy needle is in position to obtain the biopsy sample.

5. The method of claim 1, wherein marking the projected aperture as a planned biopsy location comprises marking the projected aperture with a first color.

6. The method of claim 5, further comprising changing the marking of the projected aperture to a second color after the biopsy sample has been obtained.

7. The method of claim 1, further comprising marking the transverse ultrasound image to identify the prostate gland.

8. The method of claim 7, further comprising marking the transverse ultrasound image to identify the location of the patient's prostatic urethra.

9. The method of claim 1 further comprising performing cryotherapy at the location of the biopsy comprising inserting a cryotherapy needle through the template aperture corresponding to the marked projected template aperture.

10. The method of claim 1, wherein registering the template comprises:
a) inserting two or more biopsy needles through two or more separate template apertures;
b) displaying the transverse ultrasound image;
c) marking the locations of each biopsy needle on the displayed transverse ultrasound image;
d) identifying the template apertures through which each biopsy needle was inserted; and
e) calculating the projected template aperture locations.

11. The method of claim 1, further comprising obtaining a second biopsy sample comprising:
a) acquiring a second transverse ultrasound image of the prostate in a second plane using the ultrasound probe;
b) marking a projected template aperture as a planned biopsy location on the second transverse ultrasound image;
c) inserting a second biopsy needle through the template aperture corresponding to the marked projected template aperture to the biopsy location in the second plane;
d) obtaining a biopsy sample in the second plane using the second biopsy needle; and
e) recording an identification of the aperture for the second biopsy sample.

12. The method of claim 1, further comprising:
a) marking a second projected template aperture as a planned biopsy location in the first plane;
b) inserting a second biopsy needle through the template aperture corresponding to the second marked template aperture;
c) obtaining a second biopsy sample in the first plane using the second biopsy needle; and
d) recording an identification of the template aperture used for the second biopsy sample.

13. The method of claim 1, wherein the sagittal ultrasound image is in the same plane as the biopsy needle.

14. The method of claim 1, wherein the biopsy needle is provided in a biopsy gun, further comprising:
a) advancing the biopsy needle
b) stopping insertion of the biopsy needle at a location superficial to the first plane; and
c) firing the biopsy gun to project the biopsy needle into the first plane.

15. A system for performing a transperineal biopsy of a patient's prostate comprising:
a) a transrectal ultrasound probe attached to a template grid having a plurality of apertures;
b) a visual display;
c) a biopsy control unit in electrical communication with the ultrasound probe and the visual display, wherein the biopsy control unit is a central processing unit capable of processing ultrasound image data, registering the template's location relative to the ultrasound probe, and sending data to the display to create a transverse image of the prostate including a projected template image having projected apertures; and
d) one or more biopsy needles.

16. The system of claim 15 further comprising a user interface, wherein the user interface allows a user to mark one or more projected apertures.

17. The system of claim 15 wherein the biopsy needles are sized for insertion through the apertures and into the prostate when the template is placed against the patient's perineum.

18. The system of claim 15 wherein the apertures are spaced to allow biopsy sampling of the entire prostate when the template is placed against the patient's perineum.

19. The system of claim 15 further comprising a digital storage medium.

20. A method for performing a transperineal biopsy of a patient's prostate comprising:
a) placing a template having a plurality of apertures against the patient's perineum, wherein the template is attached to a transrectal ultrasound probe;
b) acquiring a transverse ultrasound image of the prostate in a first plane using the ultrasound probe;
c) registering the template to display projected template apertures on the transverse ultrasound image;
d) marking two or more projected template apertures as a planned biopsy locations;
e) inserting biopsy needles through each of the template apertures corresponding to the marked projected template apertures
f) acquiring sagittal ultrasound images of the prostate in the plane of each biopsy needle;
g) obtaining biopsy samples using the biopsy needles;
h) recording an identification of the template apertures used for obtaining the biopsy samples;
i) acquiring a transverse ultrasound image of the prostate in a second plane using the ultrasound probe; and
j) repeating steps d) through i) in the second plane.
